(19) United States
(12) Patent Application Publication
(10) Pub. No.: US 2002/0109600 A1
(43) Pub. Date: Aug. 15, 2002

(54) Body supported activity and condition monitor

(76) Inventors: James R. Mault, Evergreen, CO (US); Neil Grimmer, San Francisco, CA (US); David Gilmore, San Francisco, CA (US); Roshi Givechi, San Francisco, CA (US); Jeanne Ragan, San Jose, CA (US); Andrzej Skoskiewicz, Menlo Park, CA (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/040,191

(22) Filed: Oct. 26, 2001

Related U.S. Application Data
(60) Provisional application No. 60/243,621, filed on Oct. 26, 2000.

(51) Int. Cl. G08B 23/00; A61B 5/02

(52) U.S. Cl. 340/573.1; 600/503

(57) ABSTRACT

A personal activity monitor adapted to be supported on the body of the user, preferably on the wrist, includes a motion sensor such as an accelerometer to generate electrical signals as a function of body motion. The monitor also includes an electronic clock and a memory for recording signals representative of the motion of the housing and their time of occurrence. User entry keys on the monitor allow the entry of signals representative of the time of food consumption and the beginning and end of exercise activity. Other activity and condition sensors may be supported on the monitor such as pulse rate detector, camera for recording images of food consumed, barcode reader and the like. The output of the memory is useful in weight control and fitness logging systems.

### RELATED APPLICATION

This application claims priority of United States Provisional Patent Application 60/243,621 filed Oct. 26, 2000 and is incorporated herein by reference.

### FIELD OF THE INVENTION

This invention relates to devices adapted to be supported on a user's body to monitor and record the user's activity and physical condition in connection with weight control and fitness systems.

### BACKGROUND OF THE INVENTION

Good health and weight control are important to a large number of people. Many people engage in conventional weight loss schemes, usually based on a restricted calorie diet. Physical activity may be included in a weight control program. A calorie management system allows a person to compare their caloric expenditure, comprising resting metabolic rate (RMR) and activity-related caloric expenditure, to their caloric intake in the form of food (including beverages). Caloric expenditure has two components, a larger contribution related to resting metabolic processes, and a smaller contribution related to the energy expended in physical activity. We may say that total energy expenditure (TEE) is the sum of resting energy expenditure (REE, a product of resting metabolic rate (RMR) and the time period of interest) and activity energy expenditure (AEE), i.e.:

TEE=REE+AEE

Calorie balance is defined in terms of the difference between TEE and the caloric intake of the person.

Successful calorie management is an important part of a weight loss program. Calorie management has two important components, recording of caloric intake and recording of caloric expenditure. Writing down foods consumed and activities performed is highly time consuming. Electronic diet calculators are known in the art, but are also often very time consuming to use. It is an object of the present invention to provide devices and systems which enable and encourage people to maintain a healthy lifestyle.

### SUMMARY OF THE INVENTION

The present invention is accordingly directed toward electronic devices which may be supported on the body of a user and include an electronic clock, a sensor for detecting body motion and levels thereof, entry means for allowing the user to record activities such as food consumption and the beginning and/or end of discrete exercise activities, sensors for detecting physiological parameters of the user relating to activity, such as heart rate, an electronic memory for storing all of these signals and the time of their occurrence, for later communication to a graphic display system. The record of activities, entries, physiological parameters, and the time of occurrence assists the user in the daily conduct of weight management or fitness programs.

One preferred embodiment of the invention, as described in detail in the following specification, takes the form of a small, lightweight electronic device which may be attached to the clothing or body of a user. The device incorporates an electronic clock and a motion sensing circuit such as an accelerometer. The electrical outputs of the clock and the motion sensor are recorded magnetically or optically on a solid-state memory which correlates time and activity. The device preferably includes pushbuttons, keys or the like which may be employed by the user to record the time of consumption of meals, the time of the beginning and/or end of physical activity such as running or bicycling and the like. These signals are similarly recorded in the solid-state memory along with their times of occurrence. The device may further include physiological sensors which contact the body to record parameters such as pulse rate, EKG, body temperature or the like. These signals may be measured on a periodic basis and recorded in the solid-state memory along with a time stamp, or the system may include level sensors and only record these physiological signals when they exceed certain levels or occur at certain times, such as at the beginning and end of exercise. The device includes means for transferring the content of the memory to a graphic display device for review by the user. This may take a variety of forms including a communication port for attaching the device to a personal computer, PDA, or printer by a cable, wirelessly, or through removal of a stick memory for insertion in the display device.

Another embodiment of the invention which will be described in detail is a wrist mounted unit in the form of a wristwatch having a display for the information recorded in the memory. This unit also preferably includes a microphone and a voice recorder to allow the user to record comments such as the nature of the exercise being engaged in, the specific foods being eaten, etc. When reproduced either by the wrist supported device or an auxiliary device to which the information is transferred, this audio file may be used along with a graphical display of the recorded information either from the device itself or an auxiliary display device, to allow the user to create a formal record of food consumption and activities for use in connection with a weight control or fitness program. The body supported device may also include other units for better recording the foods consumed. In one embodiment the device includes a barcode reader which scans the universal product codes of packaging for foods consumed so as to identify the exact nature of the food for later logging in a diet balance program. Alternatively, the body supported device may also include a camera for capturing images of foods consumed for display by the graphic processor or for transmission to a remote computer over a communications network to a computer equipped to analyze the images and determine the nutritional content for food logging purposes, or to a skilled professional who may perform the same task. The body supported unit may also include alarms for the user to suggest time for meals, exercise, or the like.

Other applications and advantages of the present invention will be made apparent through the following detailed description of preferred embodiments of the invention. The description makes reference to the accompanying drawings.

### BRIEF DESCRIPTION OF THE DRAWINGS

FIG. 1 represents a first embodiment of an activity monitor formed in accordance with the present invention;

FIG. 2 is a schematic view of the activity monitor of FIG. 1;

FIG. 3A is a front view of a wrist mounted activity monitor formed in accordance with the present invention;

FIG. 3B is a rear view of the activity monitor of FIG. 3A;

FIG. 4 is a schematic view of the activity monitor of FIG. 3A and 3B;

FIG. 5A is a schematic diagram of a health management system employing an activity monitor in communication with a personal computer system and through that personal computer to a remote computer on the Internet;

FIG. 5B is a schematic diagram of another embodiment of the health management system comprising an activity monitor in communication with a PDA and auxiliary systems through the Internet;

FIG. 6 illustrates the user breathing through an indirect calorimeter for the purpose of correlating caloric expenditure with simultaneous measurement of body activity using the monitor of the present invention; and

FIG. 7 is a graph of a printout from an activity monitor over a 24 hour period with flags for food consumption and exercise activity and a graph of the total activity calories burned during the day.

### DETAILED DESCRIPTION OF THE PREFERRED EMBODIMENTS

1: Pedometer Based Smart Activity Monitor (SAM)

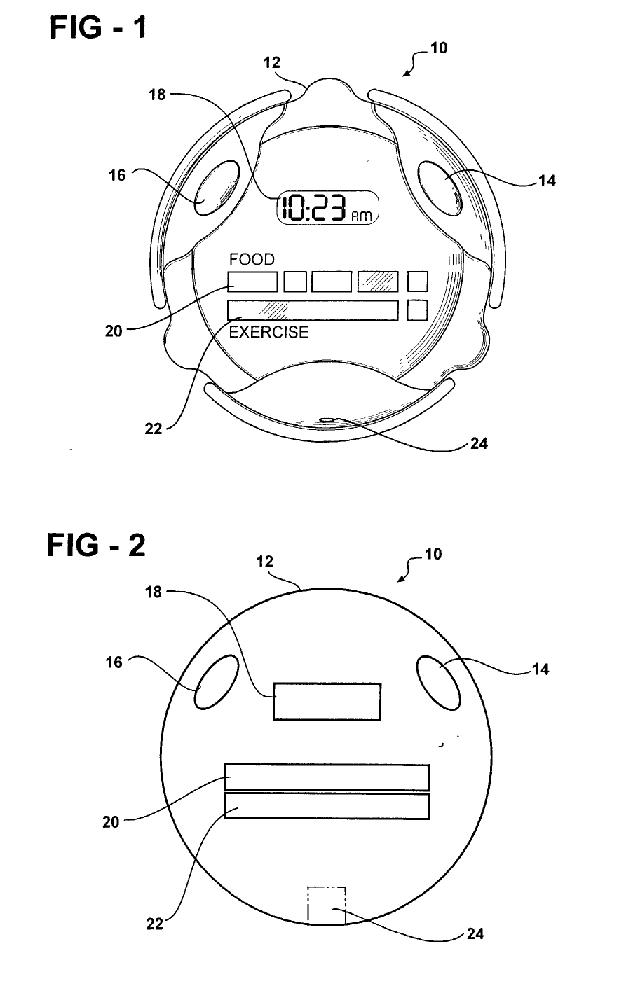

FIG. 1 shows a preferred design of a pedometer-based smart activity monitor, hereinafter sometimes referred to by the acronym SAM. Referring to the schematic of FIG. 2, the pedometer based SAM is shown generally at 10. The SAM 10 has a generally circular housing 12, an activity button 14, a food flag button 16, a time display 18, a food display 20, an exercise display 22, and an electrical connection jack 24. The housing 12 contains electronic circuitry including a clock and memory so as to function as a timepiece, record activity-related information, and record diet-related information.

In use, the SAM 10 is clipped to a belt or clothing of the user using any convenient method, for example a pin, clip, adhesive strip, hook-and-loop attachment (such as a Velcro attachment), and the like. A strap, such as a chest strap, may also be used to secure the SAM to the user's body. A skin mounted attachment may also be used, for example the SAM may be held in a clip held on the skin of the user using an adhesive layer, gel layer, or some other attachment method. (In this specification, the user is the person using the SAM, for example as part of a health-management system).

The SAM contains an internal mechanism and circuitry so as to provide an electrical signal correlated with the physical activity level of the user. Preferably, this uses a piezoelectric accelerometer based on a piezo-cantilever. Alternative accelerometer designs such as pendulum switches, conducting-liquid based switches, or other motion-sensitive switches or devices may also be used. The exercise button 14 is pressed at the commencement and the end of an exercise, so as to record the start time, end time, and duration of an activity. The time stamps are used later in building an activity log for the user. Activity levels are recorded during the activity, based on the correlation between the accelerometer signals and the activity of the user. Time data and activity level data are recorded in memory within the SAM.

The food flag button 16 is pressed when food, drinks, and other consumables are consumed. The SAM records the time of consumption, which is used later as a spur to memory when the user creates a diet log.

The activity display or exercise display 22 is used to give an indication of exercise performed during a day or other period. The bar-graph style exercise display 22 (shown in FIGS. 1 and 2) is used to provide an indication of activity levels during the day, in relation to a daily goal. The display can also be used to display the progress to the completion of an exercise. An exercise may be considered complete after a certain time has elapsed, a certain cumulative activity level has been reached, or some combination. The bar-graph style food display can be used to show the number of meals recorded. Alternatively, alphanumeric displays may be used for displays 20 and 22.

The communications jack 24 allows the SAM to communicate with another electronic device, such as a personal digital assistant (PDA), pocket-sized computer, other portable computer, wireless phone, pager, wrist mounted device, electronic book, a device containing one or more of the above functionalities, desktop computer system, or other device with computing capabilities. Preferably, a serial connector is used to interface the SAM to a computer. A wireless communications method or memory module transfer may also be used to communicate data between the SAM and the computer. This allows food-related data and activity related data to be downloaded to the computer.

In other embodiments, finger pad electrodes may be provided for bioimpedance measurements. A finger clip may be provided for pulse oximetry. A pulse rate sensor may also be provided on the rear of the housing 12, and the time display 18 used to display the pulse rate of the user. A microphone may be provided, so as to allow the user to record voice memos related to food and activity using memory within housing 12. The microphone may also be used to record heartbeats or respiratory noises for later medical diagnosis. An optical image sensor may be provided, so as to allow food and activity related images to be recorded, for example images of food consumed, printed notes or menus, the path of a walk, and the like. A barcode scanner may be provided, to allow food packages to be scanned, or other data to be entered into the SAM.

2: Wrist-mounted SAM

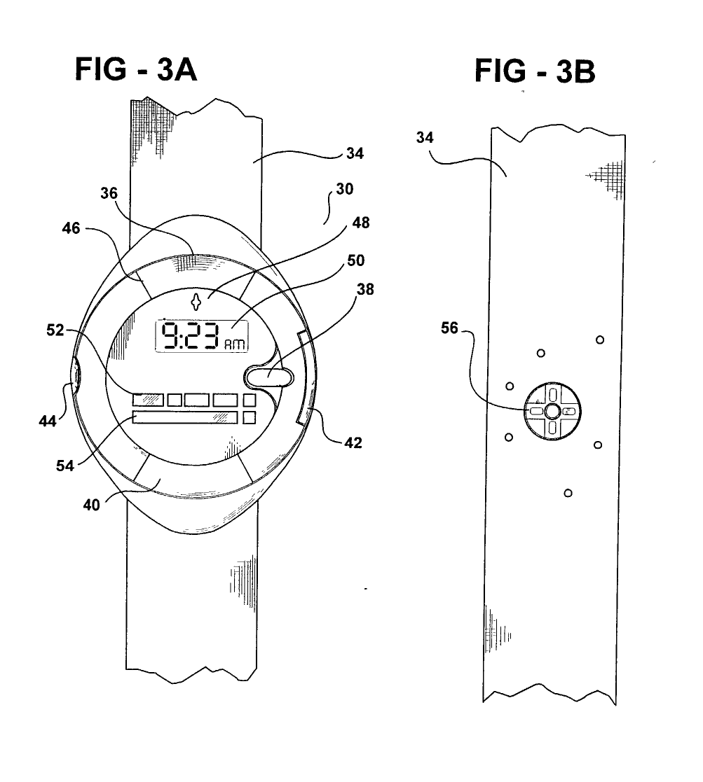

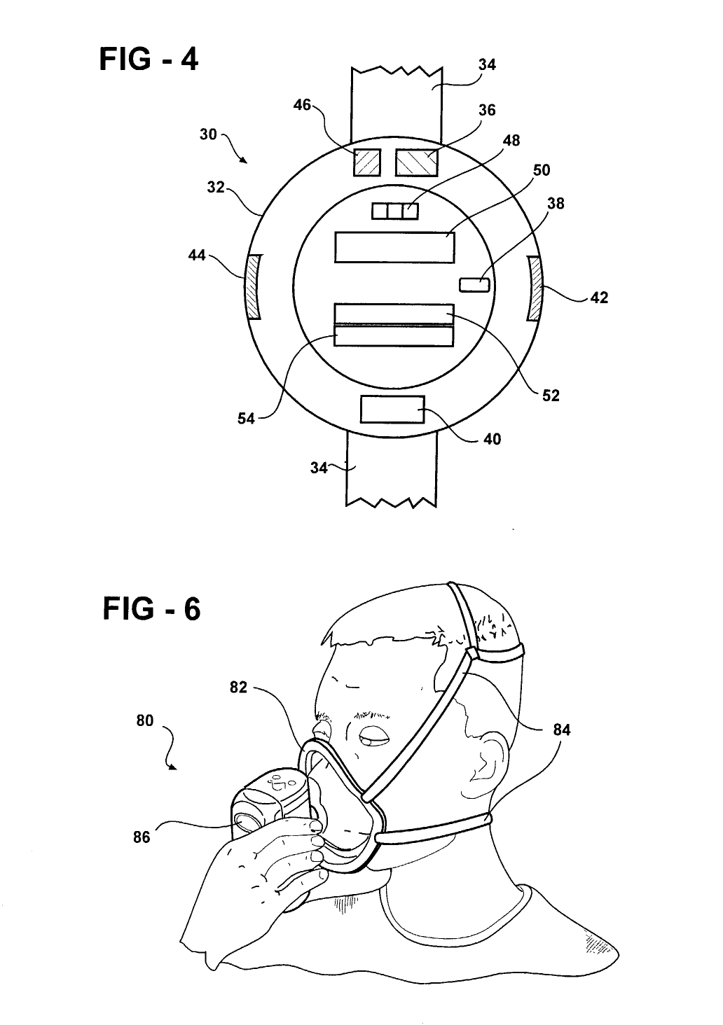

FIG. 3 shows a preferred design of a wrist-mounted SAM shown generally at 30. Referring to the schematic of FIG. 4, wrist-mounted SAM 30 has a generally rounded housing 32 in the style of a wristwatch, supported on the user's wrist by strap 34. A microphone 36 is contained within the housing 32, and provides a method of storing voice records on a memory contained within the housing 32. The SAM 30 has a mode button 38, a food flag button 40, a record/transmit button pair 42 and 44, an IR downlink port 46, a time display 50, an activity display (or exercise display) 52, and a food display 54 associated with the housing. A heart rate sensor 56 is provided on rear of the housing (as shown in FIG. 3), so as to contact the wrist of the user and provide a signal related to heart rate (or equivalently pulse rate).

The mode button 38 is used to switch between operating modes, which might include time display only, pulse rate, time from the start of an exercise, food display, activity level display, combinations of the above, and other information.

The food flag button 40 is pressed when food is eaten. The time stamp (the time at which the button was pressed) is used in creating a diet log at a later date. The time stamp data may be supplemented by recorded voice memos using the microphone. The recorded memos preferably have a time added, for assisting with diet log creation.

Data is transmitted to another device, for example a PDA or other portable computer, desktop computer system, or other electronic device, using the IR downlink port 46. This comprises an IR emitter and IR detector, so as to communicate with other devices using an IR beam. The transmit/record button pair (42 and 44) are pressed to initiate IR communication using port 46. Another wireless communications method such as the Bluetooth protocol, cable, or memory module transfer may also be used.

A heart rate sensor 56 is provided at the rear of the wrist-mounted SAM housing 32 (as shown in FIG. 4) and protrudes through a hole in strap 34 so as to contact the skin of the user. In the preferred embodiment, photo-plethysmography is used to determine the heart rate of the user. An IR source is reflected from the wrist and used to monitor the heart rate. Other techniques known in the art may be used, for example pneumatic plethysmography, in which variations in pressure are monitored, impedance cardiography, and phonocardiography. The user can also wear a chest strap adapted to measure heart rate, which communicates with SAM using a wireless method or a cable.

Other physiological monitors which may be incorporated into the wrist-mounted SAM include a blood glucose sensor, a temperature sensor, an accelerometer, and the like. Instead of or in addition to the IR port 46, the wrist-mounted SAM 30 may have an electrical connection jack for connecting the SAM to another device using a cable. The heart rate sensor may alternatively be incorporated into the strap 34. The SAM may be provided with a wireless link to a communications network such as the Internet for transfer of data to a monitoring system. The wireless link is useful for patient monitoring and athlete monitoring. The wrist-mounted SAM may also comprise the functionalities of a wireless phone, television, radio, entertainment device, Internet access device, and/or a portable computer.

3. Health Management Systems Using the SAM

One object of the present invention is to provide the user with a discreet method of making note that food was consumed. It may be impractical or embarrassing for the user to make detailed records at the time food is eaten. An advantage of the present invention is that it does not require the user to make a complete record of foods eaten at the time of consumption. By providing a food button to the SAM, a user may quickly and discreetly note the time at which they eat something. The time of consumption then acts as a cue to memory later when the complete diet log is created on the user's personal computer. Voice records may be added at the time of the meal, or at the earliest convenient time, to supplement the time stamp data.

Preferably, detailed exercise and diet logs are created using a software program running on a computing device belonging to the user. The computing device may be a personal digital assistant (PDA), pocket PC, desktop computer system, or entertainment device having computing capability. The SAM communicates with the computing device using a serial connector or other cable, wireless link such as an IR beam or the Bluetooth wireless protocol, by making an electrical interface, or by transfer of memory modules (such as a memory card, flash media, other nonvolatile memory, or powered memory module). The software is discussed in more detail in a following section. The user preferably weighs themselves at intervals, and provides this data to relevant software and databases.

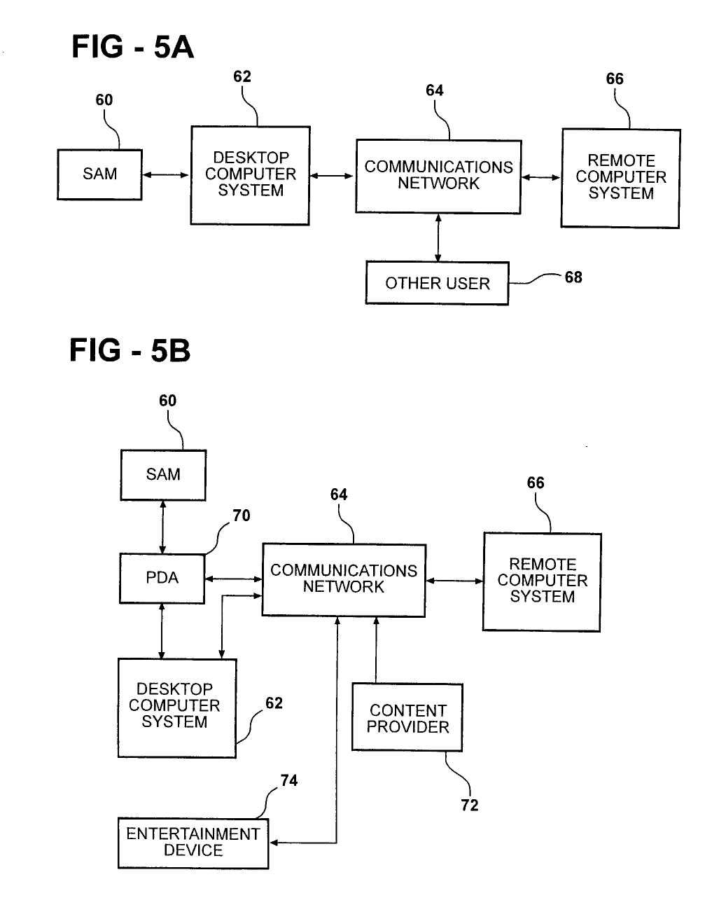

FIG. 5A shows a health management system in which SAM 60 (which may be any of the embodiments described above) communicates with desktop computer system 62. The double-headed arrows represent communications links. The computer system 62 has a link to a communications network 64, preferably the Internet. The communications network 64 is also linked to remote computer system 66 and computers belonging to other users of the health management system such as 68.

Remote computer system 66 provides a software program so as to receive data from the user and store it in a database or other memory structure. Computer system 66 also provides software so as to generate data presentations related to the user, preferably web pages viewable by the user over the Internet. Feedback, further information, resources, and support may be obtained by the user over the communications network. Progress over time may be monitored, for example by the user, employees of a business supplying the SAM, a health professional, a physician, a fitness planner, a nutritionist, or other authorized user. A support group or chat room may be provided to the user over the communications network, to assist the user in achieving weight loss goals.

FIG. 5B shows another embodiment of a health management system in which data from SAM 60 is communicated to a portable computer 70. Portable computer 70 has a link to communications network 64. Portable computer 70 also communicates with desktop computer system 62. The user also has access to entertainment device 74, which may be an interactive TV, Internet access device, and the like. Entertainment device 74 and desktop computer 62 may be combined into a unitary device. PDA 70 and SAM 60 may also be combined into a unitary device. A content provider 72 provides information which can be sent to the user over communications network 64, and viewed using devices 70, 62, or 74. Preferably, audio-visual feedback in the form of streaming video is viewed by the user on entertainment device 74. Feedback can also be in the form of electronic mail to a computing device, a web page generated by system 66 and viewable by the user, data transmitted to the user over communications network 62 so as to provide or initiate software on a computing device belonging to the user. The nature of the feedback is controlled by the status of the information stored in the database associated with remote computer system 66. For example, if weight loss goals have been met, the reward may be another episode of an entertainment series. If weight loss goals have been missed, the feedback may be in the form of diet advice or exercise tips.

An important aspect of this invention is the synchronization of data between the user's computing device or devices (in this embodiment, 70 and 62) and a remote computer system 66 using communications network 64. Data concerning the user is collected on a database on remote computer 66, which may combine diet, activity, purchase, medical, and other information generated by the SAM, diet/activity logging software, and other sources. Data is also received by the user over the network 64, for example feedback, activity suggestions, meal suggestions, and the like.

The SAM may also include a barcode reader for scanning the universal product codes (UPC), as part of a diet logging process. The SAM may also include an imaging device for capturing images of foods consumed, for the purpose of diet logging. Informal records collected by the user may be transmitted to a remote computer over a communications network, where a skilled professional, for example an employee of a health-related business, may then create the formal diet and activity logs for the user. The user may have the option of editing or enhancing the created diet log over the Internet at a later time.

Preferably, the SAM is used in a health management program in which an indirect calorimeter is provided to the user for the purpose of measuring the resting metabolic rate (RMR) of the user at intervals. Preferably, a calorimeter as described in co-pending application 09/630,398, incorporated herein by reference, is used. If the RMR falls significantly during a health management program, such as a weight loss program, the user will be encouraged to increase activity levels. Data stored on the memory of SAM, such as the expected daily activity level, will be modified to encourage a more active lifestyle.

The SAM is used to monitor physical activity levels of the user, and can provide feedback to the user if this falls below a target level. The SAM may suggest (for example through a beeping noise, flashing light, or display indication) that walking, taking the stairs, exercising, or getting up from a computer workstation is advisable. The SAM may also be used to suggest snacks, related to the time of day and the time to the next meal.

Data may be transferred from the computer back to the SAM. Food and activity records can be used to initiate or change the nature of feedback provided to the user by SAM. For example, if it appears that snacking around 11:00 a.m. is a problem in meeting a weight loss goal, the SAM can then provide feedback along the lines of suggesting the user holds out until lunch, suggesting that the user drinks water, eats a high volume low calorie snack, exercises around this time, or takes an appetite suppressant. The SAM may be provided with an alphanumeric or higher-resolution graphic display for the provision of feedback.

The SAM may also be used in cardiac recuperation programs, for example for a user recovering from heart surgery. In this case, it is often recommended that a recovering patient obtains a certain amount of physical activity. A business may provide the SAM to a user on a purchase basis, or also on a subscription basis which includes access to a website providing further feedback and support.

Embodiments of the SAM may also interact with other physiological monitors carried by the user, such as body temperature, respiration monitors, cardiac monitors, environmental sensors, and the like.

4: Calibration of SAM Using an Indirect Calorimeter The SAM is preferably used in conjunction with an indirect calorimeter. Resting metabolic rate measurements may be entered into the software used for tracking at intervals. The pedometer may also be calibrated in terms of actual caloric expenditure per unit time using a version of the GEM having a mask as the respiratory connector.

FIG. 6 shows the user breathing through an indirect calorimeter (shown generally at 80) of the type sold by HealtheTech, Inc. of Golden, Colorado under the mark HealthGem (GEM) using a mask 82 held in contact with their face using straps 84. The user measures their metabolic rate at rest, hence obtaining their resting metabolic rate (RMR). The user then performs an activity while wearing the SAM and breathing through the GEM. A correlation is formed between the increased metabolic rate measured during the activity using the GEM, and the signal from the SAM. The correlation is subsequently used to determine the actual caloric expenditure of the user based on signals from the SAM, without the necessity of breathing through the indirect calorimeter. The user's pulse rate can also be correlated with the caloric expenditure measured using an indirect calorimeter, if the SAM embodiment monitors the pulse rate of the user. The user engages in a series of activities, such walking, jogging, running, running on the spot, using a treadmill, or cycling, while wearing a SAM and breathing through the GEM. At the end of the activity, the signals from the SAM are correlated with the measured oxygen consumption using the indirect calorimeter. The SAM may also be used to identify an activity by the characteristic signature of the provided signal. For example, running may lead to a certain type of signal from the SAM, which may be recognized and the caloric expenditure of the running activity calculated. In this embodiment, the patterns provided by the SAM are be used in determining the activity levels of the user. The patterns may be unique signatures of pulse rate, accelerometer signal, position (e.g. GPS signal), and other physiological signals. The SAM may receive data from exercise machines related to activities performed. A GPS signal may locate a user within a gym, and prompt the user later to provide detailed exercise information. A personal trainer may also provide activity related data. The SAM may automatically sense when an exercise starts and stops, providing time flag data without the necessity of pushing a button. The SAM may then provide detailed activity log data, such as the time and nature of activities performed during the day, to activity logging software on a computer, without the need for data input by the user.

The SAM can provide total caloric expenditure (RMR+AEE) by receiving data from a calorimeter (such as described in co-pending application 09/630,398) indicating RMR. The RMR value is preferably updated at intervals, to compensate for the effect of RMR changes on calorie balance. Conventional weight management schemes do not compensate for RMR changes. If the RMR of the user is stored within the memory of SAM, the SAM can indicate total caloric expenditure at any time during the day. If the SAM also receives information on caloric intake (calories consumed through diet) and/or calorie targets, a calorie balance for the day can be given. For example, the SAM may include diet logging or may receive a signal from a computer on which intake is logged. Caloric intake can be estimated based on food flags and/or past intake patterns.

5. Graphic Output of SAM

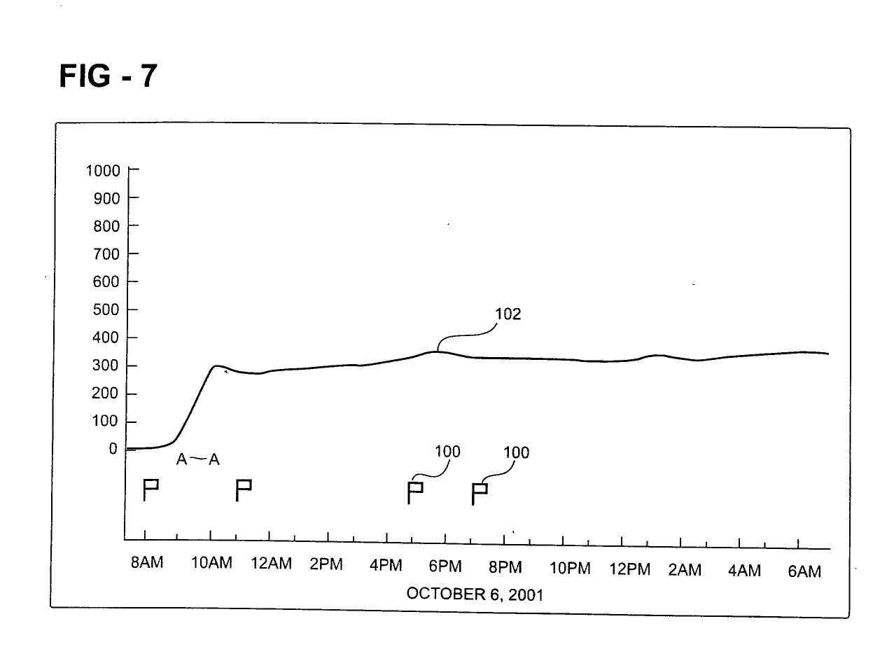

FIG. 7 illustrates a typical graphical output of SAM which may be provided on a PDA, a desktop computer or the like. The horizontal axis represents times during a 24 hour period. Flags 100 are imprinted on the graphic display at times when the user consumed food and depressed the food flag button 16. The letter A is imprinted at the time of the beginning and end of an exercise cycle resulting from pressing the exercise button 14. A graph 102 of calories expended during activity and exercise is imprinted at the top of the display. It is cumulative for the 24 hour period.

Having thus described our invention, we claim:

1. A device for monitoring and recording a user's activity for health or fitness purposes, comprising:
a housing adapted to be supported on a user's body;
a clock for generating electrical signals representative of time supported in the housing;
a motion sensor operative to generate electrical signals as a function of the user's motion, supported in the housing;
a first entry means adapted to generate an electrical signal on actuation by a user supported on the housing;
an electronic memory adapted to record said signals representative of the user's motion and the time of occurrence of signals representative of the user's motion and actuation of the first entry means supported on the housing; and
means for transferring the signals recorded in said memory to an external user display.

2. The device of claim 1 in which the motion sensor comprises an accelerometer.

3. The device of claim 1 further including a display of the current time.

4. The device of claim 1 further including sensors adapted to interact with a user's body to sense a physiological parameter.

5. The device of claim 4 wherein the physiological parameter comprises pulse rate.

6. The device of claim 4 wherein the physiological parameter comprises body temperature.

7. The device of claim 1 further including a second entry means adapted to generate an electrical signal on actuation by a user, whereby one of said entry means may record the time of consumption of food and a second of said entry means may record the time of occurrence of exercise activities.

8. The device of claim 1, further including a strap attached to the housing for securing the housing to the user's wrist.

9. The device of claim 1, further including means for securing the housing to clothing worn by the user.

10. The device of claim 1, further including a display device, adapted to receive signals from the memory to generate a graphic display containing indicia setting forth the occurrence of activity events over a period of time.

11. The device of claim 10 wherein the graphic display device constitutes a personal digital assistant.

12. The device of claim 1 further including means for communicating signals stored in said memory to a remote computer over the Internet.

13. The device of claim 1 further including a barcode reader supported on the housing and electronics adapted to store signals generated by the barcode reader in said memory.

14. The device of claim 1 further including photosensitive means for capturing images and storing them in said memory.

15. A personal activity monitor, comprising:
a housing;
means for securing the housing to the body of a user;
a clock supported in the housing;
a graphical display supported on the housing;
a motion sensor supported in the housing and operative to generate electrical signals as a function of the motion of the housing;
a plurality of user entry keys, manually actuable by the user, supported on the housing, each key being adapted to generate an electrical signal upon actuation by a user;
an electronic memory adapted to record signals representative of a user's motion and the time of occurrence of signals representative of the user's motion and of manual actuation of each of the entry keys, supported on the housing; and
means for transferring the signals recorded in said memory to an external utilization device.

16. The personal activity monitor of claim 15 wherein each of the plurality of user entry keys is associated with a separate activity of the user.

17. The personal activity monitor of claim 16 wherein at least one of the user entry keys is associated with consumption of foods and another of said user entry keys is associated with exercise activities.

18. The personal activity monitor of claim 15 further including strap means for securing the housing to the wrist of a user; and wherein said display supported on the housing displays the current time and information relating to the signals stored in the memory.

19. A personal activity monitor comprising:
a housing;
straps associated with the housing for securing the housing to the wrist of a user;
a clock supported in the housing;
a motion sensor operative to generate electrical signals as a function of the motion of the housing, supported in the housing;
a plurality of entry keys on the housing manually actuable by the user to generate electrical signals;
an electronic memory adapted to record signals representative of the motion of the housing, their time of occurrence, and actuation of each of said entry keys by the operator, supported on the housing;
a graphical display supported on the housing and adapted to display the current time and signals stored in the memory;
a pulse rate sensor supported on the housing and adapted to measure the instantaneous pulse rate of the user and generate electrical signals for provision to said memory; and
means for transferring the signals recorded in said memory to an external utilization device.

20. The personal activity monitor of claim 19 further including a camera supported on the housing and adapted to capture digital images upon actuation by the user and means for recording said digital images in the memory along with the time of their capture.

21. The personal activity monitor of claim 19 further including an audio recorder and microphone supported on the housing for use by the user in recording information relating to activities and recording the same in the memory for later reproduction.
